(19) United States
(12) Patent Application Publication
(10) Pub. No.: US 2008/0045858 A1
(43) Pub. Date: Feb. 21, 2008

(54) Device for Transcutaneous Biopsy of Tissues

(76) Inventors: Marco Tessitore, Bresso (IT); Daniele Bonara, Ponte Motta di Cavezzo (IT)

Correspondence Address: NIXON & VANDERHYE, PC, 901 NORTH GLEBE ROAD, 11TH FLOOR, ARLINGTON, VA, 22203, US

(21) Appl. No.: 10/566,982

(22) PCT Filed: Jul. 29, 2004
(86) PCT No.: PCT/EP04/08486

§ 371 Date: Mar. 22, 2007

(30) Foreign Application Priority Data
Aug. 7, 2003 (IT) MO2003AOOO230

(51) Int. Cl. A61B 10/02

(52) U.S. Cl. 600/567

(57) ABSTRACT

Device for taking a sample of biological tissue transcutaneously, comprising: a needle arrangement having a tubular-shaped body, having an end associable with a grip and being provided with an edge free at the opposite end, lamina elements movable between a neutral position wherein they lies near said tubular-shaped body and an operating position wherein they are distanced from the latter, wherein said lamina elements protrude towards said end.

The invention relates to a device for conducting a transcutaneous biopsy, i.e. a sample taken through the skin of a patient of deep tissues to be subjected to subsequent diagnostic examination, in particular hard tissues such as bone-marrow tissue.

In medical-surgical practice devices are used for transcutaneous biopsy of hard tissues comprising a hollow cylindrical needle having a proximal end provided with a grip and a distal end, possibly tapered, provided with a cutting edge. Inside the needle a stem is housed that is shaped like a steel cylindrical rod, having a proximal end provided with a grip and a pointed distal end. The stem is shapingly coupled with the cavity of the needle in such a way as to slide freely therein and has a length that is greater than the latter. In this way, by inserting the stem completely into the needle, the pointed distal end emerges from the latter. In order to conduct a bone-marrow biopsy using the device disclosed above, a health operator positions himself or herself near the patient at the anatomical region of the latter comprising the bone formation from which the sample is to be taken, for example the iliac crest. He or she then proceeds to simultaneously push and rotate the needle provided with the relative stem, through the skin and the underlying muscle until the bone is reached. The external layer of the latter, which is extremely compact and resistant, is perforated by the sharpened distal end of the stem, which enables the needle to reach the internal portion of the bone, which has a spongy structure and accommodates the marrow tissue. The operator can then extract the stem from the needle and further push the latter inside the bone-marrow tissue, still performing a pressing/rotating movement. As a consequence of this movement, the cutting distal end of the needle partially separates from the surrounding tissue a substantially cylindrical fragment constituting the bioptic sample to be taken. The latter is thus peripherally enveloped by the internal cavity of the needle and remains connected to the original tissue only due to its own portion of root that lies near the distal end of the needle.

To completely resect the sample, the operator resorts to a so-called luxation manoeuvre, consisting of making the needle swing in a manner that is substantially perpendicular to its longitudinal axis whilst simultaneously extracting the needle from the body of the patient using a rotating movement.

This procedure nevertheless has the double drawback of traumatising the patient by causing painful microfractures in the bone tissue and of being unable to ensure that the bioptic sample is effectively taken. In fact, the movements impressed on the needle may not be sufficient to produce the detachment of the portion of root of the sample, this causing the sample-taking to fail. Again, during the phase of extraction of the needle, the sample may partially emerge from the latter, being thus damaged, or it may totally emerge therefrom, thus remaining inside the body of the patient.

In both cases, the sample-taking has to be repeated in another position, with consequent further discomfort being inflicted on the patient and further work for the personnel entrusted with the task.

In an attempt to remedy these drawbacks, WO 02/07603 discloses a biopsy device in which, between the needle and the stem, a hollow cylinder is interposed the distal end of which is provided with a bendable tab arranged parallel to the longitudinal axis of the needle. When a sample of tissue remains enclosed in the distal portion of the needle, the hollow cylinder is made to slide towards the latter in such a way that the tab engages on an abutting surface with which the internal wall of the needle is provided. As a consequence, the tab is forced to bend almost at right angles, thus separating the sample from the surrounding tissue and enclosing it inside the needle. However, the bendable tab has little mechanical resistance and is not therefore suitable for taking samples of hard tissue, such as bone marrow samples. Furthermore, it is very complex and costly.

WO 02/053035 discloses a device for transcutaneous biopsy in which, between a needle having a tapered distal end and a corresponding stem a hollow rod is interposed that at its distal end is provided with a pair of slightly curved flexible laminae. When during a biopsy a sample of tissue to be sampled remains enclosed inside the distal end of the needle, by pushing the hollow rod towards the latter flexure of the laminae is obtained, which approach one another, thereby arresting the sample by friction.

WO 02/07602 discloses a device for transcutaneous biopsy comprising, interposed between a needle and a relative stem, a hollow cylindrical element provided at its distal end with a pair of flexible laminae. From one of the latter protrudes a protuberance, received in a hole obtained at the distal end of the needle. When in use a sample of tissue to be picked up is included in the distal portion of the needle the hollow body is made to slide towards the latter, causing the protuberance to disengage from the corresponding hole. The protuberance knocks against the internal wall of the needle, causing flexure of the corresponding lamina, which moves the sample by pressing it against the opposite blade.

EP 0852127 discloses a device for transcutaneous biopsy in which, between a hollow needle and stem, a pair of cannulas is interposed. A first cannula, placed inside the needle, is provided at its distal end with a pair of converging flexible laminae and receives internally a second cannula that keeps the laminae separated. When the sample to be taken is enclosed in a distal portion of the second cannula, the latter is made to slide in the direction of the proximal end of the device. In this way the flexible laminae are released, which converge together, thereby separating the sample from the surrounding tissue and arresting the former inside the device.

One drawback of EP 0852127 is its constructional complexity. Furthermore, both in WO 02/053035 and in WO 02/07602 and in EP 0852127 the laminae used to arrest the sample form a ‘pincer’ mechanism that has certain manufacturing costs and must be actuated by the operator through an appropriate procedure.

Lastly, it should be noted that to remove the sample from the devices mentioned so far, a probe normally has to be run inside the latter, which probe has a suitable length and transverse section so as to obtain the ejection of the fragment of tissue, which involves an increase in the time of intervention by the operator.

One object of the invention is to improve the devices for transcutaneous biopsy.

Another object of the invention is to improve the devices intended for conducting transcutaneous biopsies on hard tissues, in particular bone marrow tissue.

A further object is to provide a constructionally simple and easy-to-use device for transcutaneous biopsy.

A yet further object is to provide a device per transcutaneous biopsy in which it is possible to remove the sample at the root without performing complex procedures.

Another further object is to provide a device for transcutaneous biopsy equipped with a substantially reduced number of components such as to limit working time and reduce production costs.

Another yet further object is to provide a device for transcutaneous biopsy in which it is possible to remove in a substantially rapid manner the sample taken, without having to intervene with a probe.

In a first aspect of the invention, a device is provided for taking a sample of biological tissue transcutaneously, comprising: needle means having a tubular-shaped body, having an end associable with a grip and being provided with an edge free at the opposite end, lamina means movable between a neutral position wherein it lies near said tubular-shaped body and an operating position wherein it is distanced from the latter, characterized in that said lamina means protrudes towards said end.

Owing to this aspect of the invention, it is possible to create a biopsy device in rather a simple manner because the lamina means can be an integral part of the needle means. Furthermore, to actuate the lamina means it is sufficient to extract the device from the body of the patient subjected to biopsy. In fact, since the lamina means points towards the grip of the device, it tends during extraction of the latter to engage automatically in the sample, cutting a root portion thereof.

In a second aspect of the invention, a device is provided for taking a sample from a biological tissue transcutaneously, comprising: needle means having a tubular-shaped body, provided with an end associable with a grip and with an edge free at the opposite end, characterized in that said needle means comprises window means shaped in such a way that said sample can be extracted by said device through said window means.

Owing to this aspect of the invention, to extract the sample from the device it is sufficient to remove the sample through the above window means without having to use the probe.

The invention may be better understood and implemented with reference to the enclosed drawings, which illustrate an embodiment by way of non-limiting example in which:

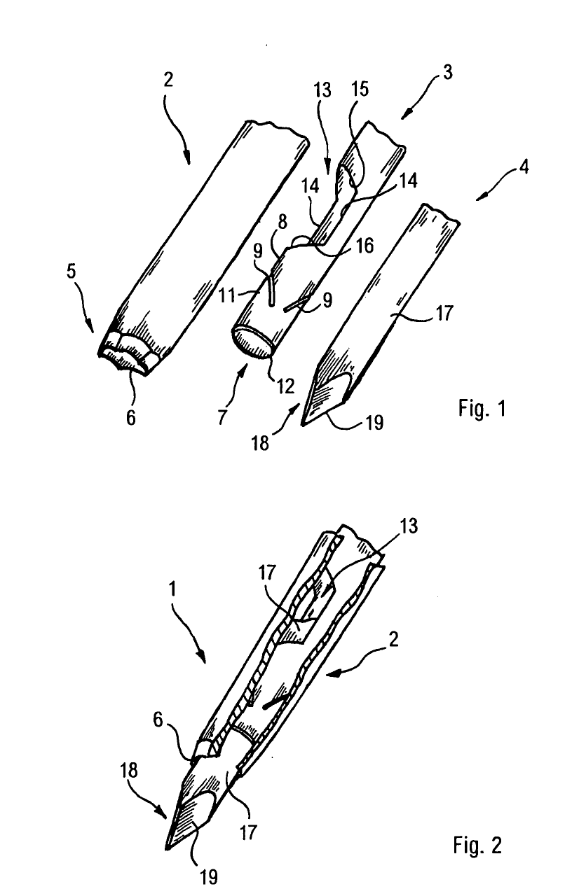

FIG. 1 is an enlarged and fragmentary perspective view of the components of a device according to the invention;

FIG. 2 is an enlarged fragmentary and partially sectioned perspective view, showing the device in FIG. 1 assembled;

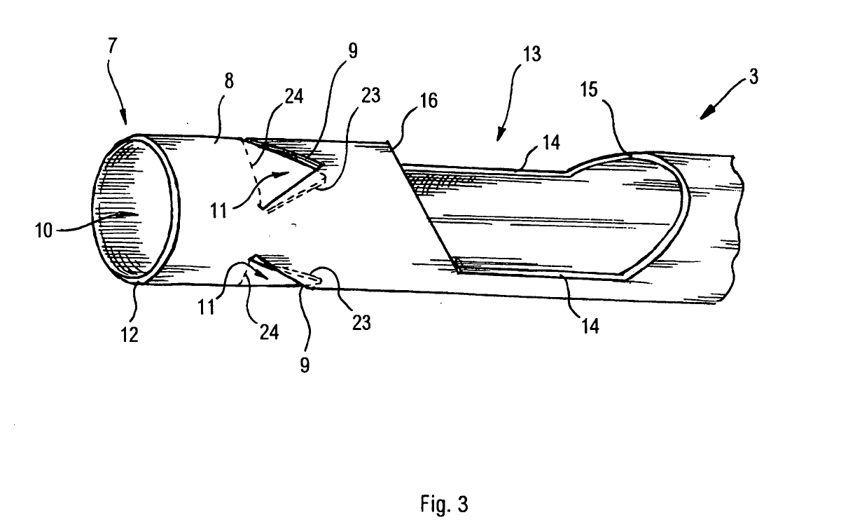

FIG. 3 is an enlarged and fragmentary perspective view of one of the components of the device in FIG. 1;

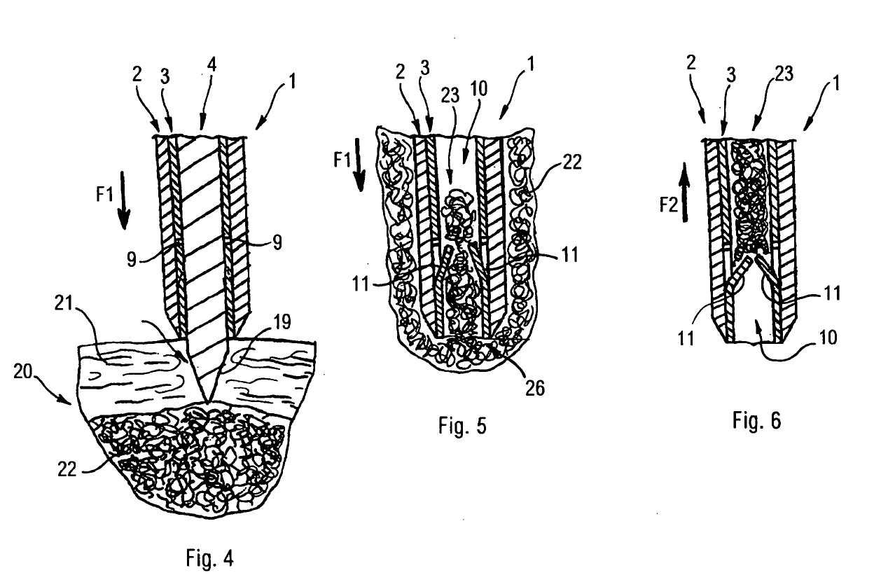

FIG. 4 is an enlarged and fragmentary longitudinal section of the device in FIG. 1, shown in an initial operating phase;

FIG. 5 is an enlarged and fragmentary longitudinal section of the device in FIG. 4, shown in a further operating phase;

FIG. 6 is an enlarged and fragmentary longitudinal section of the device in FIG. 5, shown in a yet further operating phase;

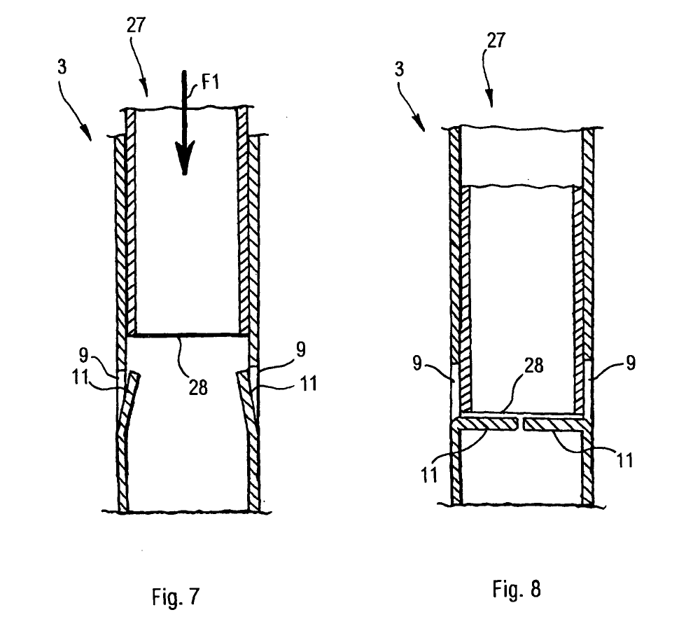

FIG. 7 is an enlarged and fragmentary schematic longitudinal section, of a further embodiment of the device according to the invention shown in an operating phase;

FIG. 8 is an enlarged and fragmentary schematic longitudinal section of the device in FIG. 7, shown in a further operating phase;

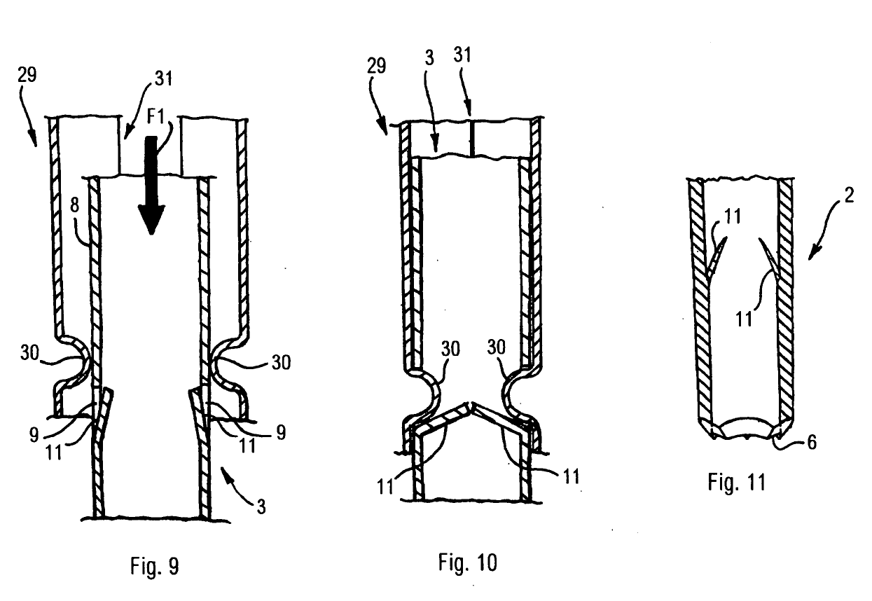

FIG. 9 is an enlarged and fragmentary schematic longitudinal section of a further embodiment of the device according to the invention, shown in an operating phase;

FIG. 10 is an enlarged and fragmentary schematic longitudinal section, of the device in FIG. 9, shown in a further operating phase;

FIG. 11 is an enlarged and fragmentary schematic longitudinal section of another yet further embodiment of the device according to the invention.

With reference to FIGS. 1 to 3, a device 1 for conducting bone-marrow biopsies comprises a hollow needle 2 with a cylindrical tubular shape, having a proximal end provided with a known operating grip, which is therefore neither shown or disclosed in detail, and a tapered distal end 5 that is provided with a cutting edge 6.

A tubular element 3, inside which a stem 4 is slidingly insertable is slidingly insertable inside the hollow needle 2 and is arranged to arrest inside itself a sample 25 taken from the patient according to a manner that will be disclosed in greater detail below. The tubular element 3 comprises a cylindrical wall 8 delimiting a tubular cavity 10 interposed between a further proximal end that is not shown, provided with an operating grip of the known type, which is not shown, and further distal end 7 provided with a circular edge 12. The cylindrical wall 8, near the further distal end 7, is provided with a release window 13, arranged to allow the extraction of the sample 25 at the end of the biopsy. The release window 13 is delimited by a pair of straight borders 14, an arched proximal border 15 and an arched distal border 16. The straight borders 14 are parallel to one another and to a longitudinal axis of the tubular element 3 and are connected with the arched proximal border 15 and with the arched distal border 16. The arched proximal border 15 is tilted towards the further proximal end of the tubular element 3, such as to delimit with the straight borders 14 a pair of equal obtuse angles, which are not shown. The arched distal border 16 is tilted in the direction of the further distal end 7, such as to form with the straight borders 14 a pair of equal obtuse angles that are not shown, the obtuse angles having the same degree as the degree of the obtuse angles formed by the arched proximal border 15 with the straight borders 14. A further embodiment of the tubular element 3 is also provided that is not shown that is made without the release window 13. In the cylindrical wall 8, between the release window 13 and the further distal end 7, three V-shaped notches 9 are obtained with the apex pointing towards the further proximal end of the tubular element 3. The notches 9 are arranged in such a way that the apices of the V are angularly spaced between themselves by about 120°. At each notch 9 a triangular lamina, or appendage, 11 is defined by a portion of cylindrical wall 8 that is near the notch 9 and points to the further proximal end of the tubular element 3. Each lamina 11 has a free cutting border 23 and a constrained border 24, indicated by a broken line, which is straight and integral with the remaining cylindrical wall 8. Each lamina 11 is furthermore slightly bent towards a longitudinal axis of the tubular element 3, in such a way as to protrude, if there is no opposing movement, inside the tubular cavity 10, as indicated by the broken lines in FIG. 3.

The prior-art stem 4 comprises a cylindrical rod 17 made with a transverse section such as to enable it to slide inside the tubular cavity 10. The rod is interposed between a yet further proximal end that is not shown provided with an operating grip that is not shown and a yet further distal end 18 comprising a penetration point 19. The length of the stem 4 is greater than the length of the tubular element 3 and of the hollow needle 2, so that the penetration point 19 protrudes outside the distal end 5 when the device 1 is assembled. FIG. 11 shows a yet further embodiment of the device 1 that does not comprise the tubular element 3, since the laminae 11 are obtained directly in the wall of the hollow needle 2. With reference to FIGS. 4, 5 and 6, when the device 1 is assembled for use, inside the hollow needle 2 the tubular element 3 is positioned and inside the latter the stem 4 is located, with the penetration point 19 protruding from the distal end 5. During this phase, the rod 17 compresses the laminae 11, preventing the latter from protruding inside the tubular cavity 10. To perform a bone-marrow biopsy on a patient, an operator, after positioning the device 1 assembled near the anatomical region housing a preselected bone formation, for example the iliac crest, makes the hollow needle 2 penetrate the underlying tissues in a penetration direction F1. As shown in FIG. 4, where for the sake of simplicity the layers of skin and muscular tissue have been omitted, when the hollow needle 2 gets near a bone 20, the penetration point 19 is used by the operator to perforate a surface layer of particularly resistant compact bone tissue 21. The stem 4 is then removed and the hollow needle 2 containing the tubular element 3 is pushed deeper into the bone 20 so as to reach an underlying spongy bone tissue 22. The latter tends to penetrate inside the tubular cavity 10 as the hollow needle 2 continues to progress into the bone 20, thereby causing the formation of the approximately cylindrical sample 25, which remains connected to the surrounding spongy bone tissue 22 only near its distal end or root 26.

During penetration of the device 1 into the spongy bone tissue 22, the laminae 11, which are no longer compressed by the rod 17, protrude slightly inside the tubular cavity 10 and are turned in a direction F2 opposite the penetration direction F1. In this way, the laminae 11 cannot be hindered and/or damaged by particularly hard tissues constituting the sample 25 inasmuch as the hard tissues will cause retraction of the laminae 11 into the thickness of the cylindrical wall 8. Furthermore, for the same reason, it is not even possible for the laminae 11 to damage the tissue forming the sample 25.

When the desired sample depth has been reached, the operator can proceed to extract the device 1 by acting in direction F2 opposite the penetration direction F1. To remove the sample 25 from the surrounding tissue 22, it is not necessary to perform any dislocating movement. In fact, by simply extracting the hollow needle 2 and the coaxial tubular element 3 in the direction F2, the laminae 11, thanks to their initial tilt, progressively engage with the sample 25. The latter presses on the laminae 11, which bend and approach one another near the longitudinal axis of the tubular element 3, tending to close the tubular cavity 10. It is therefore sufficient for the operator to rotate only slightly the device 1 around the longitudinal axis of the latter for the free cutting borders 23 of the laminae 11 to separate the root 26 of the sample 25 from the surrounding spongy bone tissue 22. The laminae 11, in their folded position, hold the sample 25 in the tubular cavity 10. Once the sample 25 has been held in the tubular cavity 10, the operator can first remove the device 1 from the body of the patient and then the tubular element 3 from the proximal end of the hollow needle 2 in such a way as to recover the sample 25 via the release window 13. In this way, the sample is extracted from the device 1 without recourse to further instrumental procedures, i.e. the operator is not obliged to slide a probe inside the tubular element 3 until the ejection of the sample 25 is obtained. Further embodiments of the device 1 are furthermore provided that enable the latter to be used effectively to perform a transcutaneous biopsy of soft tissues. The latter are in fact not sufficiently consistent to induce flexure of the laminae 11 during extraction of the device 1 from the body of the patient, as previously disclosed with reference to the biopsy of hard tissue. As a result, the free cutting borders 23 of the laminae 11 are unable to cut the root 26 of the sample 25, which cannot therefore be removed.

With reference to FIGS. 7 and 8, a further tubular element 27, fashioned in the shape of a hollow cylinder that is slidingly insertable inside the tubular element 3, is positioned in the latter in such a way that one of its distal closing ends 28 is at a certain distance from the laminae 11. The tubular element 3, which in this embodiment is provided with a distal end, which is not shown and has for example the shape of an oblique cut, can in turn be inserted into the hollow needle 2 (not shown for the sake of simplicity in FIGS. 7 to 10). The device 1 is made to penetrate into the body of a patient by an operator until it reaches a desired depth, in such a way as to cause the formation of a sample of soft tissue, not shown, which remains contained inside the apparatus 1. At this point, the operator slides the further tubular element 27 inside the tubular element 3 in direction F1 indicated by the arrow so that the closing end 28 engages with the laminae 11 bending them towards a longitudinal axis of the device 1. The laminae 11, by flexing, resect the root of the sample, which is not shown, isolating the latter from the surrounding tissue, which is not shown. The sample thus remains enclosed inside the device 1, and can thus be easily extracted together with the latter from the body of the patient.

In another embodiment shown in FIGS. 9 and 10, as an alternative to the further tubular element 27, a yet further tubular body 29 is provided that is shaped in such a way as to be slidingly interposable between the hollow needle 2 and the tubular element 3. The yet further tubular body 29 distally comprises three protuberances 30 reciprocally angularly spaced by approximately 120° and having their convexity turned towards the cylindrical wall 8 of the tubular element 3. Owing to a longitudinal incision 31 obtained in the wall of the yet further tubular body 29, the latter can be forced against the cylindrical wall 8. In this way, by positioning the yet further tubular element 30 at a certain distance from the notches 9, each protuberance 30 is applied outside the cylindrical wall 8. In use, after a sample of tissue, which is not shown, has been enclosed inside the device 1, the operator slides the yet further tubular body 29 in the direction F1 indicated by the arrow. In this way the protuberances 30 engage with the laminae 11, flexing them and the free cutting borders 23 of the latter resect the root that is not shown of the tissue sample. The latter, separated by the surrounding tissue, remains enclosed within the device 1 and can be removed together with the latter from the body of the patient.

1-9. (canceled)

10. Device for taking a sample of biological tissue transcutaneously, comprising: a needle arrangement comprising a tubular body, having a proximal end associable with a grip and being provided with an edge free at the opposite end, lamina elements protruding towards said proximal end and movable between a neutral position in which said lamina elements lies near said tubular body and an operating position in which said lamina elements is distanced from said tubular body, wherein said lamina elements is formed from a portion of said tubular body.

11. Device according to claim 10, wherein said needle arrangement furthermore comprises a window member shaped to enable said sample to be extracted by said device.

12. Device according to claim 10, wherein said needle arrangement comprises an outer tubular body extending externally of said tubular body.

13. Device according to claim 12, wherein said outer tubular body comprises protuberances so shaped as to move said lamina elements from said neutral position to said operating position.

14. Device according to claims 10, and further comprising an inner tubular body slidably insertable inside said tubular body.

15. Device according to claim 10, wherein said lamina elements are triangle-shaped.

16. Device according to claim 10, wherein said lamina elements are angularly spaced between one another by about 120°.

17. Device according to claim 10, wherein said lamina elements are defined by notches that are obtained in said tubular body.

18. Device according to claim 10, wherein said window member has a perimeter defined by a pair of straight margins that are connected at a pair of ends by an arched proximal border and at a pair of opposite ends by an arched distal border.
